(19) United States
(12) Patent Application Publication
(10) Pub. No.: US 2023/0221252 A1
(43) Pub. Date: Jul. 13, 2023

(54) METHODS AND DEVICES FOR CHARACTERIZING NANOVESICLES AND BOUND OR ASSOCIATED TARGETS THEREOF

(71) Applicant: National University of Singapore, Singapore (SG)

(72) Inventors: Xingje Wu, Singapore (GB); Haitao Zhao, Singapore (SG); Huilin Shao, Singapore (SG)

(21) Appl. No.: 17/759,820

(22) PCT Filed: Jan. 29, 2021
(86) PCT No.: PCT/SG2021/050040

§ 371 Date: Jul. 29, 2022

(30) Foreign Application Priority Data
Feb. 3, 2020 (SG) 10202000975U

(51) Int. Cl. G01N 21/64; G01N 33/543; G01N 33/574

(52) U.S. Cl. CPC G01N 21/6428; G01N 33/54346; G01N 33/57446; G01N 2021/6439; G01N 2021/6432

(57) ABSTRACT

The invention relates to methods for detecting and/or characterising a nanovesicle in a sample or a method of detecting a target that is bound or associated with said nanovesicle, wherein the sample is brought into contact with nanoparticles that are capable of binding on the surface of nanovesicle and form, in situ, a nanoshell that surround said nanovesicle. In a preferred embodiment, the nanovesicle is exosome labelled with fluorescent probes and the nanoparticles are gold nanoparticles (AuNP). The invention also relates to a kit or microfluidic chip for performing such methods, as well as a method of determining the prognosis of a cancer in a subject by performing such methods.

### FIELD

The invention described herein relates generally to the field of biotechnology. In particular, the invention relates to methods for detecting and/or characterising a nanovesicle or a method of detecting a target that is bound or associated with said nanovesicle. The invention also relates to a kit or microfluidic chip for performing such methods.

### BACKGROUND

Exosomes have recently emerged as a promising circulating biomarker. Distinguished by their biophysical and biomolecular composition, exosomes are nanoscale membrane vesicles (diameter 30-150 nm) actively released by a variety of mammalian cells, and most notably by dividing cancer cells. Exosomes contain a rich trove of molecular contents, either as inherited constituents from the parent cells or as membrane-associated molecules, that include proteins, nucleic acids, lipids as well as various modifications. As a robust messenger of intercellular communication, exosomes play an important role in mediating disease progression. Cancer cells, for example, actively produce and utilize exosomes to promote tumor growth. Exosomes are released most abundantly by rapidly dividing cancer cells. Exosomal contents not only mediate intercellular communication, but also condition the microenvironment to facilitate cancer metastasis. This orchestrated release and functional activities highlight the clinical potential of exosomes as a more reflective circulating biomarker.

Despite such clinical potential, direct and specific analysis of exosomes in native biofluids remains technically challenging, especially for clinical translation. In particular, clinical biofluids are compositionally heterogeneous, and contain nanoscale vesicles as well as abundant non-vesicle, free molecules. Current detection of the exosome population from this complex mixture relies primarily on either biophysical or biochemical characterization, performed in an independent or sequential manner. In biophysical preparation, vesicles of characteristic size could be isolated through conventional ultracentrifugation or advanced sorting strategies; however, these approaches require extensive processing, face contamination with other similarly sized protein aggregates, and lack biomolecular confirmation of vesicle identities. On the other hand, biochemical assays generally use affinity enrichment to capture and measure vesicles based on common exosomal markers. Such methods tend to miss vesicle subpopulations, and/or are susceptible to interference by biochemically identical but differentially organized molecular targets (e.g., non-vesicle, free protein antigens).

Accordingly, it is generally desirable to overcome or ameliorate one or more of the above mentioned difficulties.

### SUMMARY OF INVENTION

Disclosed herein is a method for detecting and/or characterising a nanovesicle in a sample, the method comprising the step of:

a) contacting a sample with nanoparticles or a precursor thereof, wherein the nanoparticles or precursor are capable of binding onto the surface of a nanovesicle and form, in situ, a nanoshell that surrounds said vesicle, and
b) irradiating the sample and measuring the optical signals of the sample to detect and/or characterise the nanovesicle in the sample.

Also, disclosed herein is a method for detecting one or more targets that are bound or associated with a nanovesicle in a sample, the method comprising the step of:

a) sequentially or simultaneously contacting a sample with nanoparticles, or a precursor thereof and one or more fluorescent molecular probes, wherein the nanoparticles or precursor are capable of binding onto the surface of said nanovesicle and form, in situ, a nanoshell that surrounds said nanovesicle, and wherein the one or more fluorescent molecular probes are capable of specifically binding to one or more targets that are bound or associated with the nanovesicle and provide a unique emitting fluorescence wavelength for each said target, and
b) irradiating the sample and measuring the emitted fluorescence in order to detect the one or more targets that are bound or associated with the nanovesicle, wherein the detection involves identifying an enhanced fluorescence quenching of the unique emitted fluorescence for each said target.

Also, disclosed herein is a microfluidic chip for performing a method as defined herein.

Also, disclosed herein is a kit for performing a method as defined herein.

Also, disclosed herein is a method of determining the prognosis of a cancer in a subject by simultaneously detecting or characterising one or more targets that are bound or associated with nanovesicles in a sample from the subject and are indicative of the nature of the cancer, the method comprising:

a) sequentially or simultaneously contacting a sample with nanoparticles, or a precursor thereof and one or more fluorescent molecular probes, wherein the nanoparticles or precursor are capable of binding onto the surface of said nanovesicles and form, in situ, a nanoshell that surrounds said vesicle, and wherein the one or more fluorescent molecular probes are capable of specifically binding to one or more targets that are bound or associated with the nanovesicles and provide a unique emitting fluorescence wavelength for each said target, and
b) irradiating the sample and measuring the absorbance and/or emitted fluorescence in order to detect the one or more targets that are bound or associated with the nanovesicles, wherein the detection involves identifying an enhanced fluorescence quenching of the unique emitted fluorescence for each said target.

### BRIEF DESCRIPTION OF DRAWINGS

Embodiments of the present invention are hereafter described, by way of non-limiting example only, with reference to the accompanying drawings in which:

FIG. 1: Templated nanoplasmonics for multiparametric profiling of exosomes. (a) Schematic of the TPEX (Templated Plasmonic Exosome) platform. The technology is designed to measure exosomal markers, and comprises three functional steps. Exosomes are first labeled with fluorescent molecular probes and AuNP. While AuNP remain well-dispersed when associated with non-vesicle, free proteins, they assemble onto exosome periphery, through electrostatic interactions. Excess unbound probes and AuNP are not removed. In the presence of gold salt, the AuNP serve as seeds for in situ gold growth. The dispersed AuNP experience a small growth and a slight shift in their absorbance spectra, leading to minimal changes in the fluorescence signals of probes. The exosome-bound AuNP, on the other hand, develop into a nanoshell; this nanostructure is templated by the vesicle dimension and demonstrates a large red shift in its plasmonic resonance to effectively quench the fluorescence signal of probes bound onto the same vesicle. The TPEX fluorescence signal is thus multiparametric, for both exosomal biophysical characteristics and biomarker compositions. (b) Transmission electron micrographs of TPEX products. In the presence of free proteins, AuNP remained well-dispersed (before) and demonstrated a small particle growth after treatment with gold salt (after). When incubated with exosomes, AuNP bound to vesicle periphery (before) and developed into large spherical particles after gold growth (after). Scale bars: 20 nm. (c-d) Photographs of the microfluidic device and the smartphone-based optical detector. Absorbance and fluorescence measurements could be performed on the integrated platform through different LED sources and filter configurations. Scale bar: 1 cm.

FIG. 2: TPEX absorbance analysis. (a) Optical simulations with different-sized templates. Based on microscopy characterization of the formed TPEX nanostructures, the plasmonic resonance peaks of gold nanoshells developed on different-sized templates was simulated (left). For exosome-sized templates (30-150 nm, shaded red), the resultant plasmonic peaks locate predominantly at >600 nm. Red dotted line indicates the mean peak wavelength, formed from this range of template diameters, and locates to 750 nm. Electric field distributions at 750 nm were mapped for single AuNP (bare or particles associated with free proteins) as well as gold nanoshell (exosome-templated), formed after gold growth (right). Ø indicates particle diameter after gold growth. The simulations confirmed that nanoshells templated to exosome dimension could generate strong plasmonic resonance at 750 nm. (b) Tuning of the TPEX responsive range to template diameter. Different-sized templates with AuNP of different diameters were incubated to form gold nanoshells. The TPEX absorbance measurement (A) is defined as the ratio of absorbance at 750 nm and 540 nm, and its difference (AA) before and after gold growth. Using the 9-nm AuNP, the TPEX response range could be optimized to match exosome dimension, so as to maximize exosome-induced signals. (c) Experimental evaluation with biological samples. Exosomes derived from human colorectal adenocarcinoma (DLD-1) were spiked into vesicle-depleted FBS (dFBS), and subjected to TPEX analysis with 9-nm AuNP. In all reactions, the resultant absorbance (left) and diameter changes (right) were measured. Diameter changes were performed through dynamic light scattering analysis. Only samples containing exosomes demonstrated a large signal increment, while reactions in PBS (i.e., bare AuNP) and that in dFBS (i.e., free proteins) showed negligible changes. (d) Correlation of TPEX absorbance analysis with exosome concentration. Exosomes derived from four cell lines (DLD-1, HTC116, MKH45 and SNU484) were counted through nanoparticle tracking analysis, and evaluated by the TPEX absorbance analysis. All measurements were performed in triplicate, and the data are displayed as mean±s.d. in b-c. *P<0.05, ***P<0.0005, NS, not significant, Student's t-test. a.u., arbitrary unit.

FIG. 3: Multiplexed fluorescence analysis of exosome molecular markers. (a) TPEX fluorescence analysis. To evaluate if TPEX nanoshell can be used to quench co-localized fluorescent probes, PDA nanoparticles were prepared as well-defined size templates and the particles were conjugated with fluorescent dyes (A647). The templates were treated with TPEX reaction and the resultant changes in fluorescence (ΔF, top) and absorbance (ΔA, bottom) were measured. Both analyses showed a similar trend and demonstrated a template size-responsive range optimized for exosome diameters. (b) Assay specificity to exosome markers. Whole exosomes (derived from DLD-1) that contain CD63 (top) and free CD63 (bottom) were incubated with fluorescent aptamers (anti-CD63 and scrambled control) for TPEX measurements. Only whole exosomes showed significant signals, while free CD63 samples demonstrated negligible signals. Of the different fluorescent dyes tested (FITC, RB and A647), aptamers modified with A647 (emission 665 nm, most closely matched to TPEX absorbance 750 nm) demonstrated the largest signal difference. (c) Multiplexed profiling of exosome markers. Exosomes were incubated with different fluorescent aptamers, either individually (singleplex) or as a mixture (multiplex), for TPEX analysis. The multiplex fluorescence spectrum agreed with the singleplex spectra (top), and showed accurate marker expression profiles across cell lines (bottom). (d) Molecular detection sensitivity. The limit of detection was determined by titrating a known quantity of exosomes and measuring their associating TPEX signal for CD63. The detection limit of ELISA was independently assessed based on chemiluminescence. All measurements were performed in triplicate, and fluorescence analysis was normalized against respective sample-matched scrambled controls. The data are displayed as mean±s.d. in a, b and d. *P<0.05, **P<0.005, ***P<0.0005, NS, not significant, Student's t-test. a.u., arbitrary unit.

FIG. 4: Exosome analysis in complex background. (a) TPEX analysis of mock clinical samples. Samples were prepared by spiking exosomes, derived from six human lines into vesicle-depleted human serum. In these spiked samples, exosome marker CD63, and putative cancer markers including CD24, EpCAM and MUC1 were measured. All protein measurements of the spiked samples were performed by multiplex TPEX analysis on a microfluidic platform, as well as conventional singleplex sandwich ELISA. The analyses were compared against marker signatures of pure exosomes (obtained from exosomes before spiking). For each marker analyzed, the TPEX analysis showed a better concordance to reflect the expression trends across cell lines. (b) Correlation of TPEX measurements with pure exosome signatures. The TPEX detection showed a good correlation to the pure exosome analysis (left), while the conventional ELISA measurements performed on the same spiked samples showed a significantly poorer correlation (right). All measurements were performed in triplicate, against respective sample-matched scrambled controls. The data are assay-normalized and displayed as mean in a and as mean±s.d. in b.

FIG. 5: TPEX analysis of patient prognosis. (a) Analysis of protein markers in clinical cancer ascites (n=20; 12 colorectal cancer and 8 gastric cancer) using multiplex TPEX for measurement of vesicle-associated target markers (top) and conventional singleplex ELISA for measurement of total target markers (bottom). TPEX analysis showed different protein expression profile as compared to the ELISA analysis. (b, c) Receiver operator characteristic (ROC) curves of the TPEX (b) and ELISA (c) regression models on ascites samples of colorectal cancer (left), gastric cancer (middle), and both cancer types (right). ROC curves were constructed using individual markers or a combination of the target markers (mix). The TPEX analysis showed a higher accuracy in prognosis classification across both cancers as compared to the ELISA assay. All measurements were performed in triplicate, against respective sample-matched scrambled controls. The data are assay-normalized and displayed as mean in (a).

FIG. 6. Size and molecular characterization of extracellular vesicles. Dynamic light scattering analysis of size distribution of (a) free proteins in depleted fetal bovine serum (dFBS) and (b) extracellular vesicles derived from human colorectal adenocarcinoma cell line (DLD-1). (c) Transmission electron micrograph of DLD-1 vesicles. Scale bar: 20 nm. (d) Western blotting analysis of the vesicle lysate. The lysate was immunoblotted for exosomal markers (CD63, ALIX, HSP70, TSG101, Flotillin 1).

FIG. 7. Microscopy and spectral characterization of TPEX products. Size distribution of gold nanomaterials before (top) and after (bottom) TPEX gold growth, when incubated with (a) free proteins and (b) exosomes. All measurements were determined by transmission electron microscopy (TEM) analysis. (c-d) Absorbance analysis of the corresponding gold nanomaterials. a.u., arbitrary unit.

FIG. 8. Schematics of the microfluidic platform. Exploded view of the device. The platform was assembled from two polydimethylsiloxane (PDMS) layers, and consisted of a valve layer and a microchannel layer, to construct torque-activated valves for sequential flow control and serpentine mixers for efficient labeling, respectively.

FIG. 9. Operation of the TPEX device.

FIG. 10. Optical simulations of the TPEX gold nanostructures. (a) Electric field simulations of the formed gold nanostructures, produced from bare AuNP (9-nm AuNP only) and templated by different-sized exosomes. Electric field distributions were simulated at the wavelength of 540 nm (top) and 750 nm (bottom). (b) Absorbance simulations of the formed gold nanostructures as a function of template diameter. The bare AuNP-templated nanoparticles and exosome-templated nanoshells demonstrate strong resonance at 540 nm and 750 nm, respectively. a.u., arbitrary unit.

FIG. 11. Experimental validation with different template diameters. (a) Size distribution of various polydopamine templates, as determined by dynamic light scattering analysis. (b) Template diameter as a function of sodium hydroxide volume. (c) Experimental absorbance spectra after templated nanomaterial growth. The experimental validation agrees with the simulated absorbance. Specifically, in the absence of target template (i.e., bare AuNP), a single resonance peak was observed near 540 nm; when reacted with templates of increasing diameter, an additional resonance peak emerged at 750 nm. All measurements were performed in triplicate, and the data are displayed as mean±s.d in a-b. a.u., arbitrary unit.

FIG. 12. Characterization of different-sized AuNP. (a) Absorbance measurements of different-sized AuNP, before and after functionalization with polyethylenimine (PEI). (b) Transmission electron micrographs of different-sized AuNP, after PEI functionalization. Scale bar: 50 nm. (c) Size distributions of the prepared AuNP, as determined by TEM analysis, confirming the monodispersity of the preparations. a.u., arbitrary unit.

FIG. 13. TPEX assay on exosomes and free proteins. (a) Zeta potential and (b) hydrodynamic diameters were measured of exosomes and free proteins (dFBS), under different experimental conditions (+, present; −, absent). All measurements were performed in triplicate, and the data are displayed as mean±s.d.

FIG. 14. Extracellular vesicles isolated from various cell origins. Extracellular vesicles obtained from colorectal cancer cells (a) DLD-1, (b) HCT116 and gastric cancer cells (c) MKN45, (d) SNU484. All vesicles were characterized with nanoparticle tracking analysis.

FIG. 15. TPEX absorbance analysis of exosomes. (a) Absorbance spectra of different exosome counts after TPEX reactions. Exosomes derived from DLD-1 cell lines were quantified through nanoparticle tracking analysis and subjected to TPEX reactions. (b) TPEX absorbance sensitivity. The limit of detection was determined by titrating a known quantity of exosomes and measuring their associated TPEX absorbance changes. All measurements were performed in triplicate, and the data are displayed as mean±s.d. in b. a.u., arbitrary unit.

FIG. 16. Synthesis of aptamers with branched fluorescence. Step 1: Acryl modification of aptamer with bis-acrylate molecule through aza-michael addition. Step 2: Addition of 4 arm-PEG to the modified aptamer by reacting primary amine with acryl group. Step 3: Labeling of the peglated aptamer with fluorophores through ester conjugation. After each step, the modified aptamers were purified from excess reagents through size-selective filtration (molecular cutoff, 3,000).

FIG. 17. Performance evaluation of fluorescent aptamers. (a) Anti-CD63 aptamers were prepared with branched fluorescence (3 dyes) or with a single fluorescent molecule (1 dye). The aptamers for TPEX reactions were used with exosomes, and the absorbance (top) and fluorescence (bottom) changes were measured. While both aptamer preparations showed comparable absorbance changes, the 3-dye preparation demonstrated a better fluorescence signal. All measurements were performed against respective, scrambled control aptamers. (b) Limit of detection of 1-dye aptamer. Exosomes were diluted and measured with 1-dye, anti-CD63 aptamer. All measurements were performed in triplicate, and the data are displayed as mean±s.d. *P<0.05, ***P<0.0005, NS, not significant, Student's t-test. a.u., arbitrary unit.

FIG. 18. TPEX analysis with antibodies and miRNA probes. (a) TPEX analysis with different fluorescent antibodies (top) showed accurate protein marker expression profiles across cell lines as compared to ELISA analysis (bottom). (b) TPEX analysis with different fluorescent DNA probes against miRNA targets (top) showed accurate miRNA marker expression profiles across cell lines as compared to PCR analysis (bottom). All measurements were performed in triplicate. Analysis was normalized against respective sample-matched IgG isotype control antibodies or scrambled controls in a and b, respectively.

FIG. 19. Smartphone-based detector. (a) Optical spectra of different LED sources. (b) Configurations of the smartphone-based detector, for absorbance and fluorescence detection, respectively. (c) Correlation of TPEX measurements by the smartphone-based detector and commercially available plate reader. The smartphone-based detector showed good performance correlation to the commercial reader (R²=0.9945). All measurements were performed in triplicate, and the data are displayed as mean±s.d. in c. a.u., arbitrary unit.

FIG. 20. Comparison of CD63 expression levels in clinical samples. (a) Vesicle counts of clinical ascites samples were determined by nanoparticle tracking analysis. (b) TPEX analysis of CD63 in the clinical samples. (c) ELISA analysis of the total CD63 proteins in the clinical samples. TPEX analysis of CD63 could better reflect vesicle counts, as determined by gold-standard nanoparticle tracking analysis, while ELISA analysis of total CD63 proteins showed a poor concordance to the counts.

### DETAILED DESCRIPTION

Disclosed herein is a method for detecting and/or characterising a nanovesicle in a sample, the method comprising the step of:

a) contacting a sample with nanoparticles or a precursor thereof, wherein the nanoparticles or precursor are capable of binding onto the surface of a nanovesicle and form, in situ, a nanoshell that surrounds said vesicle, and
b) irradiating the sample and measuring the optical signals of the sample to detect and/or characterise the nanovesicle in the sample.

In one embodiment, the formation of the nanoshell induces a localized plasmonic resonance and/or increased optical absorbance in the infrared region.

In one embodiment, the spectral properties (absorbance) of the nanoshell are tuned to distinguish the extracellular vesicle dimension and the respective vesicle counts.

Without being bound by theory, the inventors have developed a platform/method to enable multi-parametric molecular profiling of vesicles through the simultaneous evaluation of biophysical as well as biomolecular composition of the same vesicles—directly in native clinical biofluids. Named “Template Plasmonics for Exosomes” (TPEX), the technology utilizes the formation of gold nanoshells, assembled and grown on vesicles in situ, to achieve specific analysis of exosomal biomarkers. For biophysical-selectivity, the nanoshell formation is templated by vesicle membrane and tuned to distinguish exosome dimensions. For biomolecular-selectivity, through matched and localized energy transfer, the nanoshell's unique plasmonic signature can quench fluorescent probes only if they are target-bound on the same vesicle. The resultant optical signals (i.e., absorbance and fluorescence) can enable multi-selective analysis of diverse exosomal biomarkers (e.g., proteins and miRNAs), but remain unresponsive to non-vesicle, free molecular targets. When implemented on a microfluidic, smartphone-based sensor, the TPEX technology can achieve rapid and multiplexed analysis of exosomal targets with superior performance (1 μl of sample in 15 min). The inventors further applied the developed platform to examine native clinical ascites samples. The technology not only revealed exosomal biomolecular signatures against complex biological background, but also showed that the exosomal subpopulation of biomarkers, as compared to the total biomarkers, could more accurately differentiate cancer patient prognosis.

The method as referred to herein may comprise contacting a sample with nanoparticles or a precursor thereof, wherein the nanoparticles or precursor are capable of binding onto the surface of a nanovesicle and form, in situ, a nanoshell that surrounds said vesicle. For instance, in an embodiment the in situ formation of the nanoshell may be catalysed by adding both nanoparticles and a precursor to said nanoparticles such as a metallic salt (eg a gold or silver salt).

As used herein, the term “nanoparticle” refers to particles having a particle size on the nanometer scale, less than 1 micrometer. For example, the nanoparticle may have a particle size up to about 50 nm. In another example, the nanoparticle may have a particle size up to about 40 nm. In another example, the nanoparticle may have a particle size up to about 30 nm. In another example, the nanoparticle may have a particle size up to about 20 nm. In another example, the nanoparticle may have a particle size up to about 10 nm. In another example, the nanoparticle may have a particle size up to about 6 nm. In one embodiment, the gold nanoparticles have a diameter range of between 1-4 nm, 2-6 nm, 3-7 nm, 4-8 nm, 5-9 nm, 6-10 nm, 7-11 nm, 8-12 nm, 9-13 nm, 10-14 nm 11-15 nm, 12-16 nm, 13-17 nm, 14-18 nm, 15-19 nm or 16-20 nm.

The nanoparticles may be of a plasmonic material. Alternatively, the nanoparticles may be coated with a plasmonic material. In one embodiment, the nanoparticles are metallic nanoparticles. The metallic nanoparticles may be made a metal such as gold, silver or titanium or may be an alloy of different metals. In one embodiment, the metallic nanoparticles are gold nanoparticles. In one embodiment, the gold nanoparticles have a diameter range of between 7-11 nm. In an alternative embodiment, the nanoparticle comprises an organic polymer.

In one embodiment, the precursor is a metallic salt that, together with nanoparticles, is capable of forming a nanoshell that surrounds a nanovesicle. In one embodiment the metallic salt is gold salt. The metallic salt may be in solution prior to contact with the sample.

As used herein, a “nanovesicle” may refer to a naturally occurring or synthetic vesicle that includes a cavity inside. The nanovesicle may comprise a lipid bilayer membrane enclosing contents of an internal cavity. A nanovesicle may include a liposome, an exosome, extracellular vesicle, microvesicle, apoptotic vesicles (or apoptotic body), a vacuole, a lysosome, a transport vesicle, a secretory vesicle, a gas vesicle, a matrix vesicle, or a multivesicular body. A nanovesicle may have a dimension of about 1000 nm or less, about 900 nm or less, about 800 nm or less, about 700 nm or less, about 600 nm or less, about 500 nm or less, about 450 nm or less, about 400 nm or less, about 350 nm or less about 300 nm or less, about 250 nm or less, about 240 nm or less, about 230 nm or less, about 220 nm or less, about 210 nm or less, about 200 nm or less, about 190 nm or less, about 180 nm or less, about 170 nm or less, about 160 nm or less, about 150 nm or less, about 140 nm or less, about 130 nm or less, about 120 nm or less, about 1 10 nm or less, about 100 nm or less, about 90 nm or less, about 80 nm or less, about 70 nm or less, about 60 nm or less, about 50 nm or less, about 40 nm or less, about 30 nm or less, about 20 nm or less, or about 10 nm or less.

In one embodiment, the nanovesicle is an exosome. The term “exosome” refer to a vesicle that is shed by eukaryotic cells, or budded off of the plasma membrane, to the exterior of the cell. Exosomes can be heterogeneous in size with diameters ranging from about 10 nm to about 5000 nm.

Disclosed herein is a method for detecting one or more targets that are bound or associated with a nanovesicle in a sample, the method comprising the step of:

a) sequentially or simultaneously contacting a sample with nanoparticles or a precursor thereof and one or more fluorescent molecular probes, wherein the nanoparticles or precursor are capable of binding onto the surface of said nanovesicle and form, in situ, a nanoshell that surrounds said nanovesicle, and wherein the one or more fluorescent molecular probes are capable of specifically binding to one or more targets that are bound or associated with the nanovesicle and provide a unique emitting fluorescence wavelength for each said target, and
b) irradiating the sample and measuring the emitted fluorescence in order to detect the one or more targets that are bound or associated with the nanovesicle, wherein the detection involves identifying an enhanced fluorescence quenching of the unique emitted fluorescence for each said target.

In one embodiment, the optical properties of the fluorescent molecular probes are matched to spectral-compatibility of the nanoshell to enhance the detection signal.

In one embodiment, the optical properties of the fluorescent molecular probes are matched to spectral-compatibility of the nanoshell to distinguish biomarkers which reside within or associate with different-sized extracellular vesicles.

The target that is to be detected may be bound or associated with a nanovesicle in the sample. The target may be referred to as a “biomarker”. The target may, for example be a nucleic acid, lipid, protein, peptide, metabolite, or glycopeptide that is bound or associated with a nanovesicle. In one embodiment, the target is a nucleic acid (such as an RNA). In one embodiment, the target is a protein such as a membrane protein or membrane-associated protein. In one embodiment, the target is a lipid. In one embodiment, the target is a metabolite. The target as referred to herein may also include modified proteins, nucleic acids, lipids and metabolites. In one embodiment, the method as defined herein is capable to distinguishing between the different types of target that may be bound or associated with a nanovesicle.

In one embodiment, the target is a cancer biomarker. The cancer biomarker may, for example, be CD24, EpCAM or MUC1.

In one embodiment, the target is an exosome biomarker. The exosome biomarker may be CD63.

The term “nucleic acid”, as described herein, can be RNA or DNA, and can be single or double stranded, and can be, for example, a nucleic acid encoding a protein of interest, a polynucleotide, an oligonucleotide, a nucleic acid analogue. Such nucleic acid sequences include, for example, but are not limited to, nucleic acid sequence encoding proteins, for example that act as transcriptional repressors, antisense molecules, ribozymes, small inhibitory nucleic acid sequences, for example, but not limited to, RNAi, shRNAi, siRNA, micro RNAi (mRNAi), antisense oligonucleotides etc.

The terms “protein” and “polypeptide” are used interchangeably and refer to any polymer of amino acids (dipeptide or greater) linked through peptide bonds or modified peptide bonds. Polypeptides of less than about 10-20 amino acid residues are commonly referred to as “peptides.” The polypeptides of the invention may comprise non-peptidic components, such as carbohydrate groups. Carbohydrates and other non-peptidic substituents may be added to a polypeptide by the cell in which the polypeptide is produced, and will vary with the type of cell. Polypeptides are defined herein, in terms of their amino acid backbone structures; substituents such as carbohydrate groups are generally not specified, but may be present nonetheless.

In one embodiment, the method involves contacting the sample with nanoparticles in excess required to form the nanoshell.

The one or more fluorescent molecular probes may be capable of specifically binding to one or more targets that are bound or associated with the nanovesicle. This may provide a unique (or specific) emitting fluorescence wavelength for each said target, which allows each said target to be distinguished from one another.

The fluorescent molecular probe may be a nucleic acid, aptamer, antibody or small molecule. The fluorescent molecular probe may a molecular probe such as nucleic acid, aptamer, antibody or small molecule that is linked to a fluorescent dye. Examples of a fluorescent dye to be used for fluorescent labeling include fluorescent dyes having fluorescein, rhodamine, coumarin, Cy, EvoBlue, oxazine, carbopyronin, naphthalene, biphenyl, anthracene, phenenthrene, pyrene, carbazole, or the like as a backbone, or derivatives of such fluorescent dyes. Examples of fluorescent dyes include, but are not limited to, fluorescein, rhodamine B and Alexa Fluor 647.

The term “aptamer” refers to an oligonucleotide that can conform in three-dimensions to bind another molecule with high affinity and specificity. Aptamers are usually identified by selecting them from a large random sequence pool, but natural aptamers also exist in riboswitches. Aptamers can be broadly classified as either nucleic acid (DNA or RNA) aptamers, which consist of (usually short) strands of oligonucleotides, or peptide aptamers, which consist of a short variable peptide domain, attached at both ends to a protein scaffold. Aptamers, like peptides generated by phage display or monoclonal antibodies (MAbs), are capable of specifically binding to selected targets and, through binding, block or otherwise alter the function of the target molecule to which they bind. Aptamers are typically identified by an in vitro selection process (such as, e.g., SELEX) from pools of random sequence oligonucleotides. Aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family).

In one embodiment, the aptamer comprises a nucleic acid sequence selected from the group consisting of SEQ ID NO: 1, SEQ ID NO: 2, SEQ ID NO: 3 and SEQ ID NO: 4. The aptamer may be conjugated to a fluorescent dye.

By “antibody” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present invention include polyclonal and monoclonal antibodies as well as their fragments (such as Fab, Fab′, F(ab′)2, Fv), single chain (scFv) and domain antibodies (including, for example, shark and camelid antibodies), and fusion proteins comprising an antibody, and any other modified configuration of the immunoglobulin molecule that comprises an antigen binding/recognition site. An antibody includes an antibody of any class, such as IgG, IgA, or IgM (or sub-class thereof), and the antibody need not be of any particular class.

In one embodiment, the antibody is selected from the group consisting of an anti-CD63 antibody, an anti-CD24 antibody, an anti-EpCAM antibody and an anti-MUC1 antibody.

The method may comprise irradiating the sample and measuring the emitted fluorescence in order to detect the one or more targets that are bound or associated with the nanovesicle. The detection may involve identifying an enhanced fluorescence quenching of the unique emitted fluorescence for each said target.

The term “enhanced fluorescence quenching” may refer to increase in the level of fluorescence quenching as compared to a reference. The reference may, for example, be an emitted fluorescence in the absence of one or more targets that are bound or associated with the nanovesicle.

In one embodiment, the sample is a sample that has been obtained from a subject. In one embodiment, the subject is a subject suffering from cancer.

The term “sample” may refer to any sample derived from or containing cells, organisms (bacteria, viruses), lysed cells or organisms, cellular extracts, nuclear extracts, components of cells or organisms, extracellular fluid, media in which cells or organisms are cultured in vitro, blood, plasma, serum, gastrointestinal secretions, urine, ascites, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, pleural fluid, nipple aspirates, breast milk, external sections of the skin, respiratory, intestinal, and genitourinary tracts, and prostatic fluid.

A sample can be a biological sample which refers to the fact that it is derived or obtained from a living organism. The organism can be in vivo (e.g. a whole organism) or can be in vitro (e.g., cells or organs grown in culture). A “biological sample” also refers to a cell or population of cells or a quantity of tissue or fluid from a subject. Most often, a sample has been removed from a subject, but the term “biological sample” can also refer to cells or tissue analyzed in vivo, i.e., without removal from the subject. Often, a “biological sample” will contain cells from a subject, but the term can also refer to non-cellular biological material, such as non-cellular fractions of blood, saliva, or urine. The biological sample may be from a resection, bronchoscopic biopsy, or core needle biopsy of a primary, secondary or metastatic tumor, or a cellblock from pleural fluid. In addition, fine needle aspirate biological samples are also useful. In one embodiment, a biological sample is ascites. Biological samples also include explants and primary and/or transformed cell cultures derived from patient tissues. A biological sample can be provided by removing a sample of cells from subject, but can also be accomplished by using previously isolated cells or cellular extracts (e.g. isolated by another person, at another time, and/or for another purpose). Archival tissues, such as those having treatment or outcome history may also be used. Biological samples include, but are not limited to, tissue biopsies, scrapes (e.g. buccal scrapes), whole blood, plasma, serum, urine, saliva, cell culture, or cerebrospinal fluid. The samples as referred to herein may have been proceed for purification or enrichment of nanovesicles such as exosomes.

The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, and esophageal cancer. In one example, the cancer is colorectal or gastric cancer.

As used herein, the term “subject” includes any human or non-human animal. In one embodiment, the subject is a human. The term “non-human animal” includes all vertebrates, e.g., mammals and non-mammals, such as non-human primates, sheep, dog, cow, chickens, amphibians, reptiles, etc.

In one embodiment, the characterisation of the one or more targets comprises measuring the level of the one or more targets that are bound or associated with nanovesicles.

Disclosed herein is a microfluidic chip for performing a method as defined herein. The microfluidic chip may comprise one or more microfluidic channels (e.g. 2, 3, 4, 5, 6, 7, 8, 9, 10 or more microfluidic channels). The use of microfluidics in the methods as described herein significantly reduces the amount of sample needed for detection.

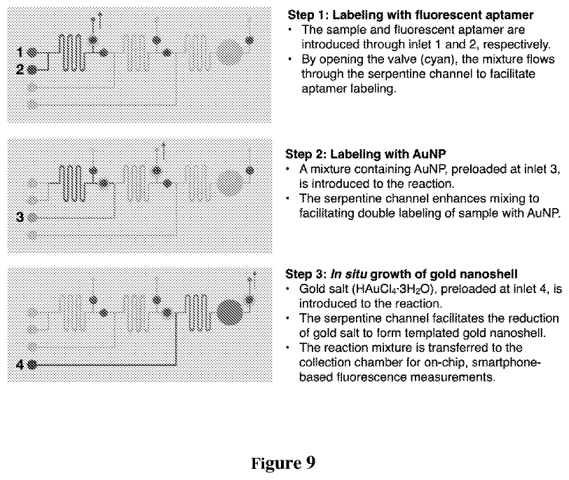

The microfluidic channels can have multiple functions. Each channel can be fluidically independent (e.g. having its own fluid inlet and outlet). The microfluidic channels can, for example, be used to facilitate mixing of the sample with the fluorescent molecular probe, or mixing of the sample with the nanoparticles. These steps may be performed concurrently or sequentially. Finally, the microfluidic channels may also be used to facilitate in situ growth of a nanoshell around a nanovesicle. The microfluidic channels may further be used to transfer the reaction mixture to a collection chamber for on-chip or smartphone-based fluorescence measurements. In one embodiment, the microfluidic chip is one that is as shown in FIG. 9.

Disclosed herein is a kit for performing a method as defined herein. The kit may comprise reagents such as fluorescent molecular probes for binding to one or more targets that are bound or associated with a nanovesicle, nanoparticles and precursor that are capable of binding onto the surface of a nanovesicle and forming, in situ, a nanoshell that surrounds the nanovesicle. The kit may further comprise buffers, instruction manual, and the like. The kit may also provide a microfluidic chip as defined herein for performing a method disclosed herein.

Disclosed herein is a method of determining the prognosis of a cancer in a subject by simultaneously detecting or characterising one or more targets that are bound or associated a nanovesicle in a sample from the subject and are indicative of the nature of the cancer, the method comprising:

a) sequentially or simultaneously contacting a sample with nanoparticles, a precursor and one or more fluorescent molecular probes, wherein the nanoparticles and precursor are capable of binding onto the surface of said nanovesicle and form, in situ, a nanoshell that surrounds said nanovesicle, and wherein the one or more fluorescent molecular probes are capable of specifically binding to one or more targets that are bound or associated with the nanovesicle and provide a unique emitting fluorescence wavelength for each said target, and
b) irradiating the sample and measuring the absorbance and/or emitted fluorescence in order to detect the one or more targets that are bound or associated with the nanovesicle, wherein the detection involves identifying an enhanced fluorescence quenching of the unique emitted fluorescence for each said target.

The term “prognosis” as referred to herein refers to a prediction of the probable course and outcome of a clinical condition or disease. A prognosis of a patient is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of months, on average, that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

In one embodiment, the cancer is colorectal or gastric cancer. The method as defined herein may refer to determining of the prognosis of a cancer such as colorectal or gastric cancer in a subject. The method may comprise obtaining a sample from the subject. The sample may, for example, be clinical cancer ascites from the subject.

In one embodiment, a subject suffering from cancer may be determined to have good prognosis with an expected (or predicted) overall survival of more than ten months. In another embodiment, a subject suffering from cancer may be determined to have a poor prognosis with an expected (or predicted) overall survival of less than five months.

In one embodiment, the one or more targets comprises a target selected from the group consisting of CD63, CD24, EpCAM and MUC1.

In one embodiment, the method comprises treating a subject. The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

Disclosed herein is a method of detecting a cancer in a subject by simultaneously detecting or characterising one or more targets that are bound or associated a nanovesicle in a sample from the subject and are indicative of the presence of the cancer, the method comprising:

a) sequentially or simultaneously contacting a sample with nanoparticles or a precursor thereof and one or more fluorescent molecular probes, wherein the nanoparticles or precursor are capable of binding onto the surface of said nanovesicle and form, in situ, a nanoshell that surrounds said nanovesicle, and wherein the one or more fluorescent molecular probes are capable of specifically binding to one or more targets that are bound or associated with the nanovesicle and provide a unique emitting fluorescence wavelength for each said target, and
b) irradiating the sample and measuring the absorbance and/or emitted fluorescence in order to detect the one or more targets that are bound or associated with the nanovesicle, wherein the detection involves identifying an enhanced fluorescence quenching of the unique emitted fluorescence for each said target.

In one embodiment, the method comprises determining the likelihood of the presence (or absence) of a cancer in a subject.

In one embodiment, the method further comprises treating a subject found to have cancer.

Disclosed herein is a method of treating a cancer in a subject by simultaneously detecting or characterising one or more targets that are bound or associated a nanovesicle in a sample from the subject and are indicative of the presence of the cancer, the method comprising:

a) sequentially or simultaneously contacting a sample with nanoparticles or a precursor thereof and one or more fluorescent molecular probes, wherein the nanoparticles or precursor are capable of binding onto the surface of said nanovesicle and form, in situ, a nanoshell that surrounds said nanovesicle, and wherein the one or more fluorescent molecular probes are capable of specifically binding to one or more targets that are bound or associated with the nanovesicle and provide a unique emitting fluorescence wavelength for each said target,
b) irradiating the sample and measuring the absorbance and/or emitted fluorescence in order to detect the one or more targets that are bound or associated with the nanovesicle, wherein the detection involves identifying an enhanced fluorescence quenching of the unique emitted fluorescence for each said target, and
c) treating the subject found to have cancer.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### EXAMPLES

Methods

Cell Culture

All human cancer cell lines were obtained from American Type Culture Collection. DLD-1, HCT116, GLI36vIII were grown in Dulbecco's modified essential medium (Hyclone) supplemented with 10% fetal bovine serum (FBS, Gibco) and 1% penicillin-streptomycin (Gibco). MKN45, SNU484, and PC9 were cultured in RPMI-1640 medium (Hyclone) supplemented with 10% FBS and 1% penicillin-streptomycin. All cell lines were tested and free of mycoplasma contamination (MycoAlert Mycoplasma Detection Kit, Lonza, LT07-418).

### Exosome Isolation and Quantification

Cells at passages 1-15 were cultured in vesicle-depleted medium (containing 5% vesicle-depleted, dFBS) for 48 h before vesicle collection. All media containing extracellular vesicles were filtered through a 0.2-μm membrane filter (Millipore), isolated by differential centrifugation (first at 10,000 g and subsequently at 100,000 g). For independent quantification of vesicle concentration, the nanoparticle tracking analysis (NTA) system (NS300, Nanosight) was used. Vesicle concentrations were adjusted to obtain ˜50 vesicles in the field of view to achieve optimal counting. All NTA measurements were done with identical system settings for consistency.

### Synthesis and Characterization of AuNP

All chemicals used for synthesis and modification were purchased from Sigma-Aldrich, unless otherwise stated. AuNP were prepared by a sodium citrate approach. Briefly, different-sized AuNP were synthesized by varying the amount of sodium citrate in the reaction. In a typical synthesis, to prepare AuNP with diameter 9 nm, 50 ml of sodium citrate tribasic dehydrate (0.6 mg/ml) was heated to boil. Subsequently, 250 μl of gold (III) chloride trihydrate (HAuCl₄·3H₂O, 20 mg/ml) was quickly injected into the boiling solution and reacted for 30 min to produce AuNP. After cooling to room temperature, 9 ml of the prepared solution was mixed with 1 ml of polyethylenimine (PEI, 10% in water) to replace the surface ligand on AuNP. The PEI-coated AuNP were then centrifuged at 20,000 g for 1 h to remove excess reactants, and resuspended and kept at 4° C. for future use. For AuNP characterization, particle core diameters were measured with transmission electron microscopy (JEOL 2010F). Hydrodynamic diameter and zeta potential of AuNP were determined with Zetasizer Nano ZS instrument (Malvern). 3×14 measurement runs were performed. Z-average diameter and polydispersity were analyzed. For every measurement, the autocorrelation function and polydispersity index were monitored to ensure sample quality for size determination. Optical absorbance of AuNP was measured spectroscopically (Tecan).

### Synthesis and Characterization of PDA Particles

To prepare different-sized PDA nanoparticles as target templates, 1 ml of dopamine hydrochloride (0.5 mg/ml in water) was mixed with a varying volume of sodium hydroxide solution (4 mg/ml, volume varied from 1-50 μl). The mixture was incubated at 25° C. under stirring condition for 12 h to produce PDA particles with well-defined diameters. All particles were stored at 4° C. for subsequent use. Particle size distribution was determined by dynamic light scattering analysis, as described above. To label PDA particles with respective fluorophores (e.g., fluorescein, rhodamine B and Alexa Fluor 647), fluorescent dyes dissolved in dimethyl sulfoxide were added to the PDA solution (0.5 mg/ml). The mixture was incubated at 25° C. for 12 h, before sample purification. Fluorescence intensity was measured through a microplate reader (Tecan).

### Preparation of Fluorescent Aptamers

All aptamer sequences used in this study can be found in Table 1. DNA sequences, modified with a primary amine group at the 3′ end, were purchased from Integrated DNA Technologies and dissolved in water to a final concentration of 10 μM. To enhance the fluorescence performance of the aptamers, a single aptamer sequence was labelled with three fluorescent molecules. Specifically, 100 μl of aptamer solution was reacted with 10 μl of N,N-methylenebisacrylamide (1 mM) for 12 h at 37° C. to produce acrylated aptamer. This purified reaction was added to an excess of 4-arm poly(ethylene glycol) with free amines (4 arm-PEG2K—NH2, molecular weight=2000, 100 μM, 40 μl) for 12 h at 37° C. Finally, fluorescent dyes (e.g., Alexa Fluor 647) were conjugated to the free amines on the peglated aptamers. After each reaction step, the modified aptamers were purified by a centrifugal filter (Amicon, molecular cutoff=3000) to remove excess reactants. Purified fluorescent aptamers were kept at −20° C. for future use.

TABLE 1
List of aptamers, antibodies and sequences used.
Aptamers

| | |
|---|---|
| CD63 | CACCCCACCTCGCTCCCGTGACACTA |
| | ATGCTA-NH₂ (SEQ ID NO: 1) |
| CD24 | TATGTGGGTGGGTGGGCGGTTATGC |
| | TGAGTCAGCCTTGCT-NH₂ |
| | (SEQ ID NO: 2) |
| EpCAM | CACTACAGAGGTTGCGTCTGTCCCAC |
| | GTTGTCATGGGGGG |
| | TTGGCCTG-NH₂ (SEQ ID NO: 3) |
| MUC1 | GCAGTTGATCCTTTGGATACCCTG |
| | G-NH₂ (SEQ ID NO: 4) |

Antibodies

| | |
|---|---|
| Anti-CD63 | BD Biosciences, clone H5C6 |
| Anti-CD24 | eBioscience, clone eBioSN3 |
| Anti-EpCAM | R&D Systems, clone 158206 |
| Anti-MUC1 | Fitzgerald, clone M01102909 |

miRNA

| | |
|---|---|
| 21-5p | NH₂-TCAACATCAGTCTGATAAGCTA |
| | (SEQ ID NO: 5) |
| 221-3p | NH₂-GAAACCCAGCAGACAATGTAGCT |
| | (SEQ ID NO: 6) |

Optical Simulation

Full 3D finite-difference time-domain (FDTD) simulations were performed using a commercial software package (FDTD Solutions, Lumerical). Based on transmission electron miscroscopy analysis of the formed nanostructures, the exosome-templated gold nanoshell was modeled as a core-shell structure, with a dielectric core of refractive index (RI) of 1.4(35), surrounded by a 9 nm-thick gold shell. The complex dielectric constants for gold were obtained from reference(36). In simulating the field distribution of AuNP bound to free proteins, as experimentally characterized with dFBS, AuNP with a final diameter of 14 nm after growth was modeled to attach to a 3-nm protein. A uniform mesh of 2 nm was applied in all directions. In all simulations, the formed gold nanostructures were illuminated with a plane wave from the top and the transmitted (absorbance) spectrum was recorded at the bottom. The simulated electric field distribution and absorbance spectra were used to identify the corresponding resonance peaks of nanostructures templated by exosomes and free proteins, respectively.

TPEX Absorbance Assay

To experimentally evaluate and validate the optical simulations, the TPEX assay on PDA nanoparticles of different diameters was first performed. These PDA nanoparticles were used as target templates with well-defined size distribution. Briefly, 5 μl of PDA solution were incubated with 5 μl of AuNP solution for 15 min at room temperature to enable self-assembly of AuNP on PDA surface. Without any purification, a mixture containing 10 μl of hydrogen peroxide (3%), 35 μl of PBS buffer and 40 μl of gold salt (HAuCl₄·3H₂O, 1 mg/ml) was added to this reaction. The reaction was incubated for 15 min to enable templated in situ gold growth. Absorbance spectra were recorded before and after gold growth to compare the experimental results against that of the simulations. To investigate the effect of AuNP diameter in tuning the TPEX absorbance response, the PDA nanoparticles were incubated with different-sized AuNP, before the reactions were subjected to gold growth. The 9-nm AuNP was chosen for all subsequent TPEX measurements, so as to match and maximize the TPEX responsive range to published exosome diameter. The optimized TPEX assay was further applied on biological samples. Extracellular vesicles and vesicle-depleted FBS (dFBS) were prepared through differential centrifugation, as described above. All samples were characterized by NTA and dynamic light scattering analysis. Biological samples were treated with AuNP and subjected to gold growth, as described above in the PDA reactions. Corresponding absorbance spectra, before and after gold growth, were measured spectroscopically.

TPEX Fluorescence Assay

For detection of molecular markers, the TPEX fluorescence assay was developed. The assay was optimized with fluorescent anti-CD63 aptamers. Using exosomes isolated from cell lines as well as free CD63 proteins (Proteintech), these samples were incubated with 0.5 μl of fluorescent aptamer (10 μM) for 30 min. Subsequently, 5 μl of AuNP (9 nm) was added to this reaction and incubated for 15 min. Without any purification, 10 μl of hydrogen peroxide (3%), 35 μl of PBS buffer and 40 μl of gold salt (HAuCl₄.3H₂O, 1 mg/ml) was added to this reaction, as described above. For multiplexed fluorescence detection, different fluorescent aptamers were added to the sample and incubated simultaneously before AuNP incubation. For all TPEX fluorescence measurements, a sample-matched control which was incubated with scrambled aptamers was included. Fluorescence intensities, before and after the TPEX reactions, were measured.

TPEX Analysis

Based on optical simulation and experimental validation, the TPEX absorbance and fluorescence measurements are defined as follows:

ΔA=A_{after}−A_{before}

Where A_{after}=TPEX absorbance signal (A), after AuNP incubation and gold growth A_{before}=TPEX absorbance signal (A), after AuNP incubation but before gold growth And A=A₇₅₀/A₅₄₀

where A₇₅₀ and A₅₄₀ are absorbance intensities at wavelength 750 nm and 540 nm, respectively.

ΔF=1−Fₛₐₘₚₗₑ/F_{control}

Where Fₛₐₘₚₗₑ=fluorescence intensity of the sample, incubated with target probe of distinct emission spectrum, after gold growth

F_{control}=fluorescence intensity of sample-matched control, incubated with scrambled fluorescent probe, after gold growth

TPEX Antibody and miRNA Detection

For TPEX measurement with antibodies, exosomes were isolated from various cell lines and the samples were incubated with fluorescent antibodies (anti-CD63, BD Biosciences and anti-CD24, eBioscience, 1 μg/ml). Without any purification, AuNP as well as gold salt mixture were added to this reaction, as described above, and the resultant changes in fluorescence was measured. All measurements were compared against gold-standard ELISA analysis using the same antibodies (see below for details).

For TPEX miRNA detection, whole exosomes were subjected to additional fixation and permeabilization (BD Biosciences), before being labeled with fluorescent DNA probes against miRNA targets (Integrated DNA Technologies, 10 μM). Without any purification, AuNP as well as gold salt mixture were added to this reaction, as described above, and the resultant changes in fluorescence was measured. All measurements were compared against gold-standard Taqman assays (Thermo Scientific) through polymerase chain reaction (PCR, Applied Biosystems).

Microfluidic Device Fabrication

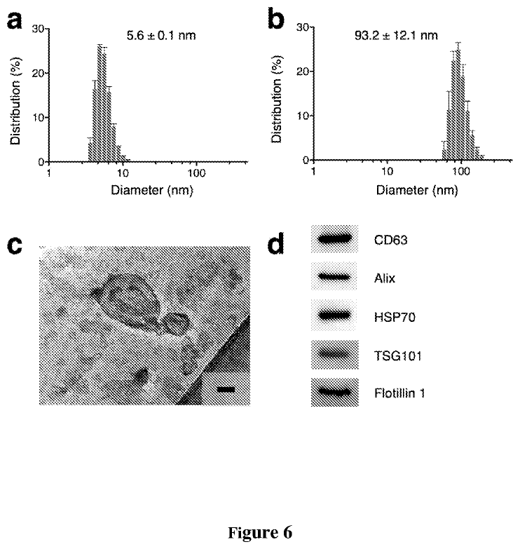

A prototype microfluidic device comprising three regions (FIG. 6) was fabricated through standard soft lithography. Briefly, 50 μm-thick cast molds were patterned with SU-8 photoresist and silicon wafers using a cleanroom mask aligner (SUSS MicroTec), and developed after UV exposure. Polydimethylsiloxane (PDMS, Dow Corning) and crosslinker were mixed at a ratio of 10:1 and casted on the SU-8 mold. The polymer was first cured at 75° C. for 30 min. Then, multiple nylon screws and hex nuts (RS Components) were positioned on the PDMS film over their respective channels and embedded in the PDMS, before a final curing step.

Microfluidic TPEX Assay

Operation steps of the microfluidic assay are illustrated in FIG. 9. In a typical procedure, 1 μl of biological sample and 0.3 μl of fluorescent aptamer solution (10 μM) were loaded into the microchannel through inlet 1 and inlet 2, respectively. This solution was mixed thoroughly in the serpentine channel to facilitate aptamer labeling of exosomal membrane biomarkers. A mixture containing 1 μl of AuNP, 2 μl of hydrogen peroxide (3%) and 8 μl of PBS buffer, preloaded at inlet 3, was introduced to the reaction and allowed to mix for 5 min in the microchannel, at a flow rate of 2 μl/min. Finally, 7 μl of gold salt (HAuCl₄·3H₂O, 1 mg/ml), preloaded at inlet 4, was added to the reaction and allowed to mix for 3 min in the microchannel. The resultant fluorescence intensity was recorded through a smartphone-based optical sensor.

Smartphone-Based Sensor

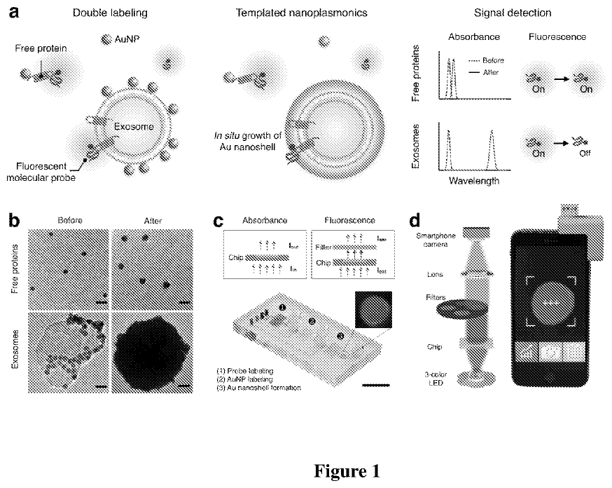

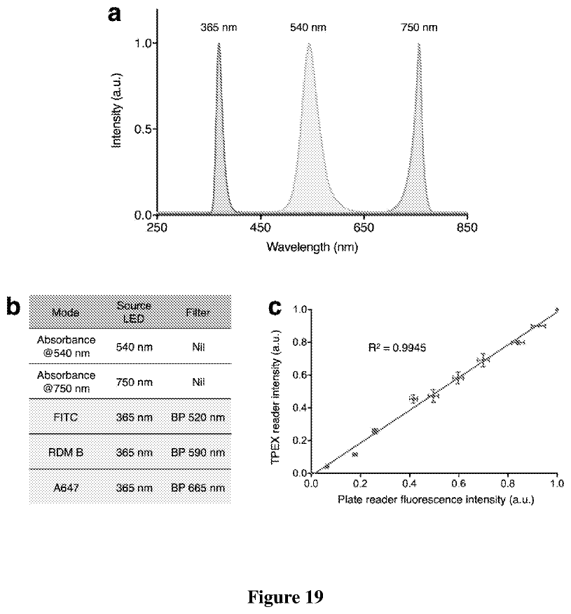

To enable smartphone analysis of the microfluidic TPEX assay, a sensor that comprised four components (FIG. 1c): a 3D-printed optical cage, a three-color LED source, three optical filters, and a magnification lens was developed. The optical cage was fabricated from a UV-curable resin (HTM 140) using a desktop 3D printer (EnvisionTEC, Aureus). The LED light source (Chaoziran S&T) was customized with three LED diodes, with central wavelengths at 365 nm, 540 nm, and 750 nm, respectively (FIG. 19a). Three bandpass filters with center wavelengths of 520 nm, 590 nm and 665 nm were used for measurements of fluorescein, rhodamine B and Alexa Fluor 647, respectively. The magnification lens (Thorlabs LA4280) was placed before the smartphone camera to improve the image quality. The assembled system measured 45 mm (width)×45 mm (length)×50 mm (height) in dimension and was equipped with two sliding slots for quick attachment to smartphones (Apple). Sensor performance was evaluated against a commercial microplate reader (Tecan) for different fluorescent dyes and intensities (FIG. 19c).

Western Blotting

Exosomes isolated by ultracentrifugation were lysed in radio-immunoprecipitation assay (RIPA) buffer containing protease inhibitors (Thermo Scientific) and quantified using bicinchoninic acid assay (BCA assay, Thermo Scientific). Protein lysates were resolved by sodium dodecyl sulfate polyacrylamide gel electrophoresis (SDS-PAGE), transferred onto polyvinylidene fluoride membrane (PVDF, Invitrogen), and immunoblotted with antibodies against protein markers: CD63 (Invitrogen), Alix (Cell Signaling), HSP70 (BioLegend), LAMP-1 (BD Biosciences), Flotillin 1 (BD Biosciences), and TSG101 (BD Biosciences). Following incubation with horseradish peroxidase-conjugated secondary antibody (Cell Signaling), enhanced chemiluminescence was used for immunodetection (Thermo Scientific).

ELISA

Capture antibodies (5 μg/ml) were adsorbed onto ELISA plates (Thermo Scientific) and blocked in PBS containing 1% BSA before incubation with samples. After washing with PBST (PBS with 0.05% Tween 20), detection antibodies (1 μg/ml) were added and incubated for 2 h at room temperature. Following incubation with horseradish peroxidase-conjugated secondary antibody (Thermo Scientific) and chemiluminescent substrate (Thermo Scientific), chemiluminescence intensity was determined (Tecan).

Transmission Electron Microscopy

Sample solutions were directly deposited onto the surface of formvar-carbon film-coated copper grid (Latech). Dried samples were imaged with a transmission electron microscope (JEOL 2010F).

Clinical Measurements

The study was approved by the National University Hospital (2016/01088), and SingHealth (2015/2479) Institutional Review Boards. All subjects were recruited according to IRB-approved protocols after obtaining informed consent. Ascites samples were collected from colorectal cancer and gastric cancer patients, centrifuged at 500 g for 10 min, and filtered through a 0.2-μm membrane filter (Millipore). All samples were de-identified and stored at −80° C. before TPEX measurements.

For clinical TPEX analysis, ascites samples were used directly. The ascites samples were incubated with fluorescent aptamers against different biomarkers and subjected the samples to TPEX reactions (i.e., AuNP incubation and in situ gold growth). For all TPEX measurements, a patient sample-matched, scrambled control was included. TPEX analysis was performed relative to this control to account for nonspecific binding of aptamers. Clinical evaluation of patient characteristics was determined independently. Specifically, patient prognosis was determined by the overall survival from the time of collection of ascites. Patients were deemed to have a good prognosis when the overall survival was more than ten months. Conversely, patients were determined to have a poor prognosis if the overall survival was less than five months. All TPEX measurements were performed blinded from these clinical evaluations.

Statistical Analysis

All measurements were performed in triplicate, and the data displayed as mean±standard deviation. Significance tests were performed via a two-tailed Student's t test. For inter-sample comparisons, multiple pairs of samples were each tested, and the resulting P values were adjusted for multiple hypothesis testing using Bonferroni correction. An adjusted P<0.05 was determined as significant. Correlation analysis was performed with linear regression to determine the goodness of fit (R²). For clinical analysis, the TPEX and ELISA measurements were used to develop multiple linear regression scoring models for the classification of disease prognosis. To avoid overfitting and evaluate performance, leave-one-out cross-validation was conducted. For a single marker, receiver operating characteristic (ROC) curves were determined from the marker expression. For multi-marker analysis, ROC curves were plotted based on the regression scorings. Statistical analyses were performed using R (v.3.5.0) and GraphPad Prism (v.7.0c).

### Example 1

TPEX Platform

The TPEX platform is designed to distinguish and measure exosomal markers (i.e., constituent and bound markers) from non-vesicle, free molecules. It consists of three functional steps: double labeling, development of templated nanoplasmonics and signal detection (FIG. 1a). In the first step, a complex biological mixture (e.g., exosomes and free proteins) is incubated with fluorescent molecular probes (e.g., aptamers) as well as gold nanoparticles (AuNP). While AuNP remain monodispersed when associated with free proteins, due to the entropy-driven formation of protein corona, they assemble onto exosome periphery, through electrostatic interactions with the exosomal membrane. Excess unbound probes and AuNP are not removed. In the next step, the AuNP serve as seeds for in situ nanomaterial growth. AuNP associated with free proteins (or unbound AuNP) experience a meager growth, and show a minimal red shift in their absorbance spectra. On the contrary, AuNP bound to exosomal surface develop into a nanoshell, templated by the vesicle dimension, to induce strong localized plasmonic resonance in the infrared region(23). The TPEX platform leverages this disparity in the resultant nanomaterial morphology and plasmonic properties to achieve simultaneous and multi-selective measurement of exosomal markers. Specifically, the spectral-compatibility of the nanoshell is templated by exosome membrane and tuned to distinguish exosome dimensions (i.e., selective for exosome biophysical properties); the enhanced fluorescence quenching of probes is observed only if they are target-bound and co-localized on the same vesicle as the formed nanoshell (i.e., selective for molecular markers). As free proteins cause minimal signal changes, the TPEX platform enables direct quantification of exosomal markers in native biofluids, obviating any purification.

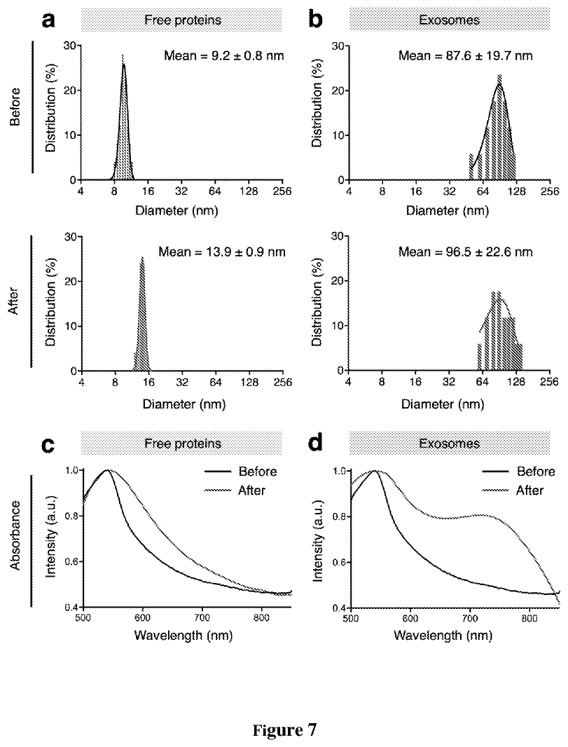

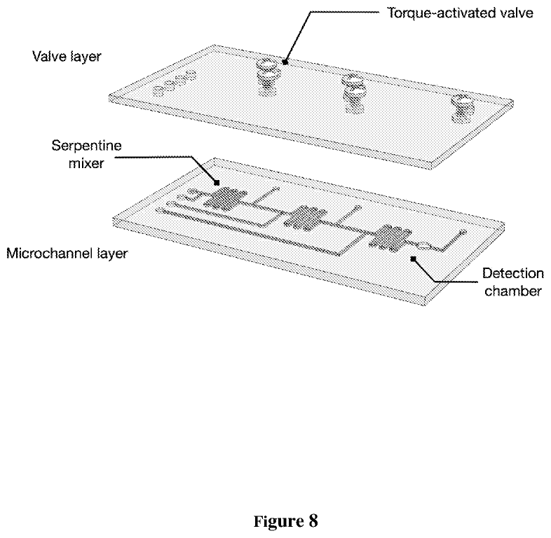

To confirm the TPEX-induced changes in nanomaterial morphology, transmission electron microscopy (TEM) analysis, before and after gold growth (FIG. 1b) was performed. In the presence of free proteins (FIG. 6a), AuNP (mean diameter=9.2 nm) remained well-dispersed and demonstrated a small particle growth after TPEX reaction. When incubated with exosomes derived from human colorectal adenocarcinoma cell line (DLD-1) (FIG. 6b-d), AuNP bound to vesicle periphery. TEM analysis further confirmed the presence of large spherical particles after nanomaterial growth, consistent with the formation of exosome-templated gold nanoshell (FIG. 7a-b). Absorbance spectra of the formed nanomaterials corresponded well with the TEM characterization (FIG. 7c-d). To facilitate TPEX measurements of complex clinical biofluids, the technology was implemented in a miniaturized microfluidic system (FIG. 1c). The device incorporates serpentine mixers for efficient labeling and torque-activated valves for fluidic control (FIG. 8) and was designed to streamline the TPEX assay operation (FIG. 9). Furthermore, the microfluidic system can be loaded onto a custom-designed, smartphone-based optical detector (FIG. 1d), which enabled absorbance and fluorescence measurements through different configurations of LED light source and filter setting. Image-based data acquisition and analysis could be achieved automatically through a smartphone interface.

Exosome-Templated Nanoplasmonics

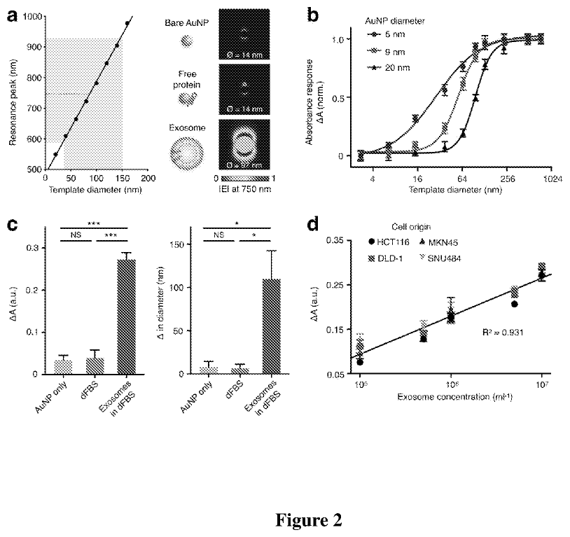

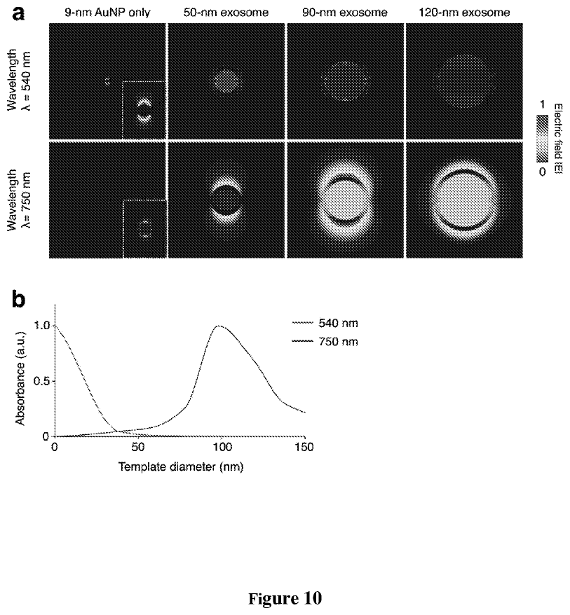

To evaluate the size effect of biomarker template on TPEX plasmonic profile, so as to optimize the technology for exosome dimension, numerical simulations was first performed for a range of template diameters (FIG. 2a). Based on TEM characterization of the formed nanostructures (FIG. 1b and FIG. 7), a 9-nm gold nanolayer was simulated to grow on the surface of exosome-sized template. The simulation results showed that for exosome diameters (30-150 nm), the resultant plasmonic resonance peaks locate predominantly at >600 nm (mean peak position at 750 nm), distinct from that formed of smaller templates (e.g., bare AuNP or AuNP associated with free proteins) (FIG. 2a). The electrical field distribution and normalized absorbance spectra further confirmed that the exosome-templated nanoshell and bare AuNP-templated nanoparticles demonstrate strong resonance at 750 nm and 540 nm, respectively (FIG. 10).

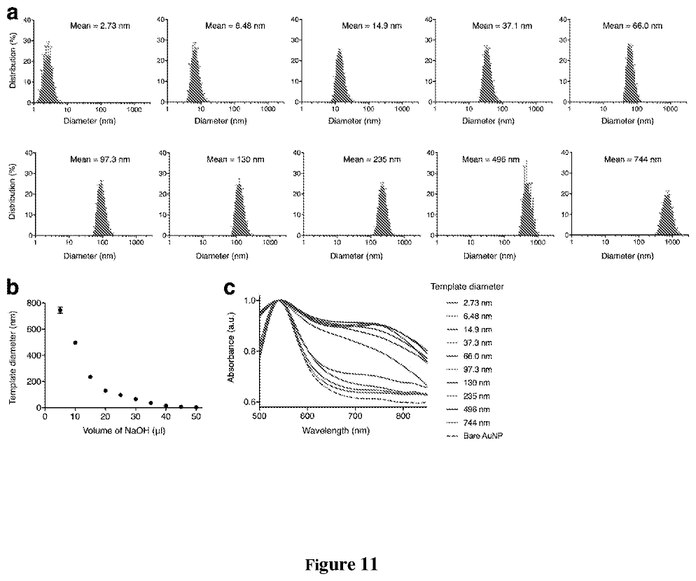

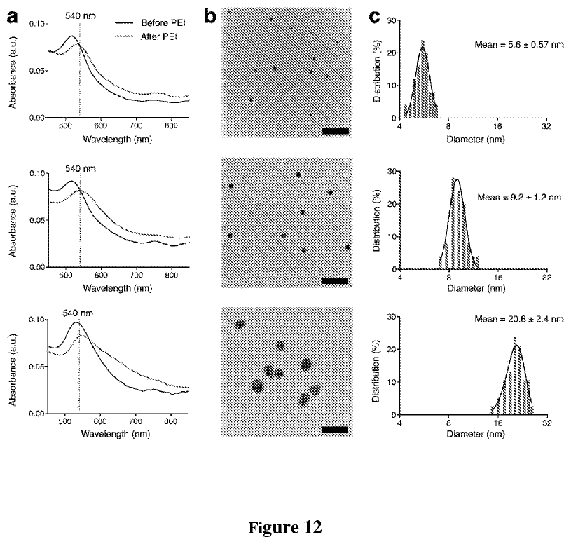

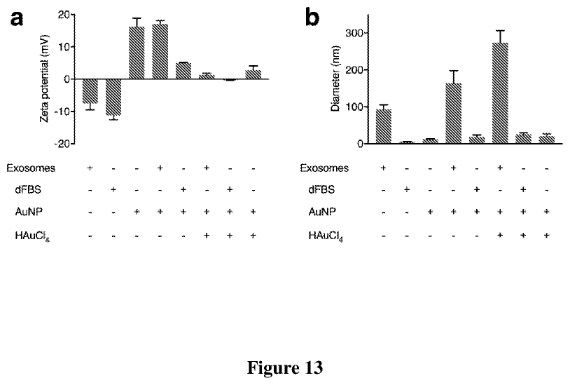

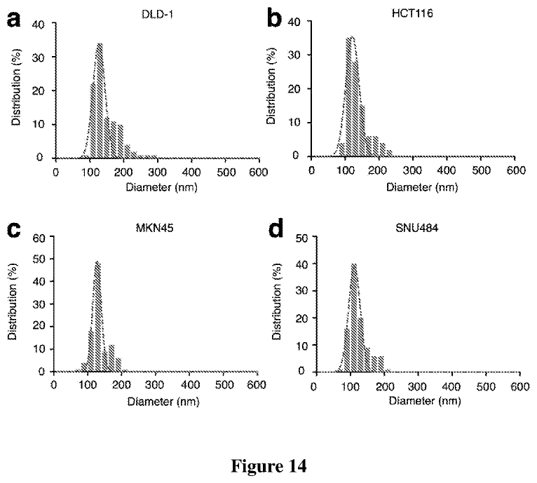

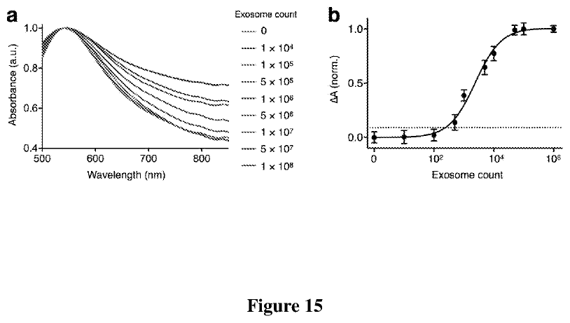

To experimentally validate the simulation results, polydopamine (PDA) nanoparticles was prepared as different-sized templates with well-defined diameter distribution (FIG. 11a-b), and these templates were incubated with AuNP (mean diameter=9.2 nm). The resultant absorbance spectra after templated nanomaterial growth confirmed the simulation results. In the absence of target template (i.e., bare AuNP), a single resonance peak was formed near 540 nm; when reacted with templates of increasing size, an additional resonance peak emerged at 750 nm (FIG. 11c). The TPEX absorbance measurement (A) was thus defined as the ratio of absorbance at 750 nm and 540 nm, and its difference (ΔA) before and after gold growth to evaluate the formation of large templated nanoshell. Interestingly, it was found that by using different-sized AuNP (FIG. 12), the responsive range of TPEX absorbance against templates of different diameters (FIG. 2b) could be fine tuned. The 9-nm AuNP was thus chosen for all subsequent TPEX measurements to match the responsive range to exosome diameter (30-150 nm), thereby maximizing exosome-induced signals and minimizing background interference from other smaller biological entities. The optimized TPEX absorbance analysis (ΔA) was further validated with biological samples. Exosomes derived from human colorectal adenocarcinoma (DLD-1) were spiked into vesicle-depleted FBS (dFBS) and subjected to the TPEX reaction (FIG. 6). The corresponding absorbance analysis reflected good selectivity for exosomes. Specifically, AA demonstrated a large increment only in the presence of exosomes, and showed negligible changes for reactions in PBS (i.e., bare AuNP) and that in dFBS (i.e., free proteins) (FIG. 2c, left). A similar selectivity was observed for the resultant changes in particle diameter, as determined by dynamic light scattering analysis, before and after gold growth (FIG. 2c, right). This good specificity of TPEX is attributed to its assay design, that exploits multiple biophysical properties of vesicles in forming distinct plasmonic profile; the negatively-charged vesicle membrane facilitates electrostatic binding of AuNP and the vesicle itself acts as a scaffold for developing size-compatible gold nanoshell whose plasmonic properties are templated by the vesicle diameter (FIG. 13). Leveraging the specificity of TPEX absorbance analysis, the system for determining exosome concentrations was evaluated. Exosomes derived from various cell origins (DLD-1, HCT116, MKN45 and SNU484, FIG. 14) were diluted to different concentrations, quantified by gold-standard nanoparticle tracking analysis, before being spiked into dFBS. Across all spiked samples tested, TPEX absorbance analysis could directly determine exosome concentrations (FIG. 15) and demonstrated a good correlation (R²=0.931) to the gold standard measurements (FIG. 2d).

Multiplexed Fluorescence Detection of Exosomal Markers

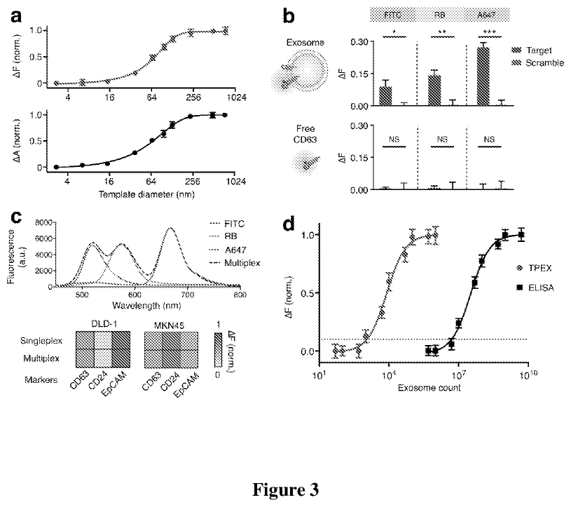

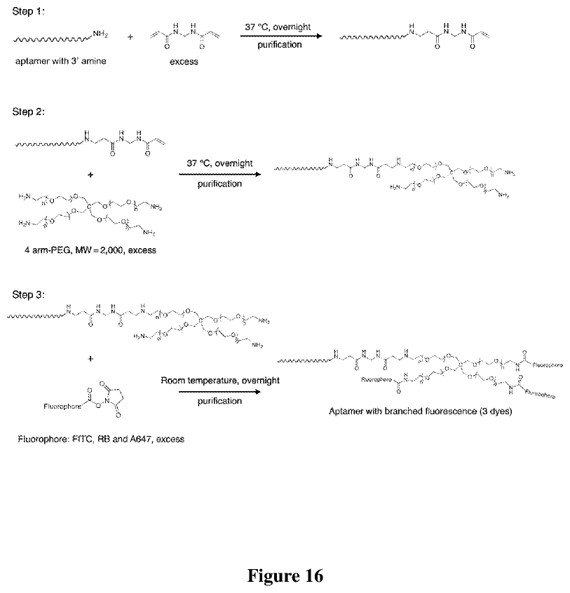

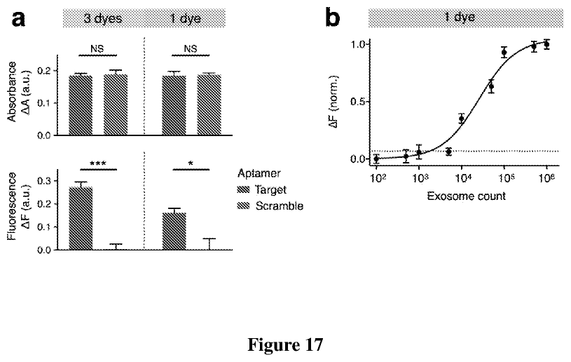

The technology was next expanded for multiplexed detection of exosome molecular markers. The plasmonic properties of the TPEX nanoshell were utilized to quench co-localized fluorescent probes. To evaluate the technology, PDA nanoparticles of various sizes were prepared, and fluorescent dyes (A647) were attached on the PDA surface. The nanoparticles were subjected to TPEX reactions (i.e., AuNP incubation and gold growth) and changes in their fluorescence intensity (ΔF) as well as absorbance signal (ΔA) (FIG. 3a) were monitored. Both analyses showed a similar trend and demonstrated a template size-responsive range that was optimized for exosome diameters. The TPEX fluorescence analysis was next applied for exosomal marker evaluation. Using CD63, a tetraspannin membrane protein found abundant in and characteristic of most exosomes, as a positive control target, two samples were prepared to evaluate the technology specificity: whole exosomes that contain CD63 (derived from DLD-1 cell line) as well as free CD63 proteins (FIG. 3b). The samples were incubated with fluorescent aptamers (anti-CD63 and scrambled control) for TPEX measurements. Each aptamer was modified with three identical fluorescent molecules (FIG. 16) to enhance its signal performance (FIG. 17). Importantly, three different types of fluorescent dyes (i.e., fluorescein/FITC, rhodamine B/RhB, Alexa Fluor 647/A647) were evaluated, selected for their distinct excitation and emission profiles, to examine the effect of resonance spectral matching for TPEX analysis. Across all fluorescent dyes tested, TPEX showed significant signals only in the presence of exosomes and displayed negligible signals to free CD63 proteins. Aptamers modified with A647, which has an emission peak (665 nm) most closely matched to the TPEX absorbance (750 nm), demonstrated the largest signal difference (FIG. 3b). Consistent with published report, these observations suggest that the TPEX fluorescence quenching is influenced by electron-transfer at the gold nanoshell surface (i.e., distance effect) as well as spectral-matching (i.e., plasmon and fluorescence).

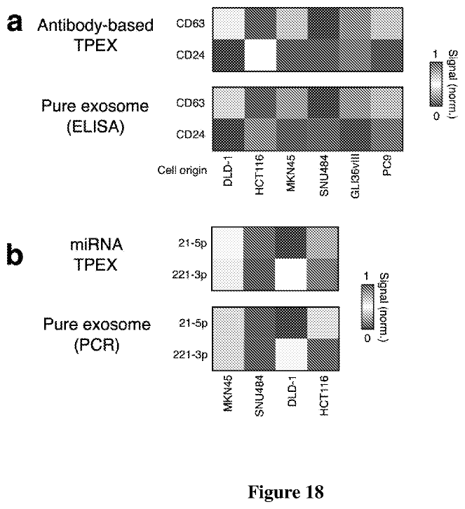

Employing different fluorescent aptamers, a multiplexed TPEX analysis for simultaneous detection of exosomal markers in a single test was developed. Exosomes derived from human cancer cells (i.e., DLD-1 and MKN45) were incubated with different fluorescent aptamers, either individually (singleplex) or as a mixture (multiplex), for TPEX measurements (FIG. 3c). The multiplex fluorescence spectrum agreed with the singleplex spectra and could accurately reveal marker expression profiles. In addition, this multiplexed TPEX assay could be adapted for protein measurements with fluorescent antibodies, and expanded for in situ analysis of miRNAs in whole exosomes (FIG. 18). The technology's molecular detection sensitivity was further determined through a titration analysis (FIG. 3d). Exosome counts were measured through nanoparticle tracking analysis. The measured TPEX response, as determined by CD63 aptamer analysis, correlated to exosome counts and established a limit of detection ˜1500 exosomes. This observed sensitivity was >10³-fold better than that of ELISA analysis.

In Situ Analysis in Complex Background

Next, the TPEX platform was evaluated to measure exosomal marker signatures against the complex biological background of native biofluids (i.e., human serum). Mock clinical samples were prepared by spiking exosomes, derived from various human lines (i.e., DLD-1, HCT116, MKN45, GLI36vIII and PC9) into vesicle-depleted human serum. Based on published literatures, the expression of the following protein markers: exosome marker CD63, and putative cancer markers including CD24, EpCAM and MUC1, was measured. TPEX analysis was performed on the spiked samples through the miniaturized microfluidic system and smartphone detection platform (FIG. 1c-d), which showed good performance correlation to commercial readers (FIG. 19).

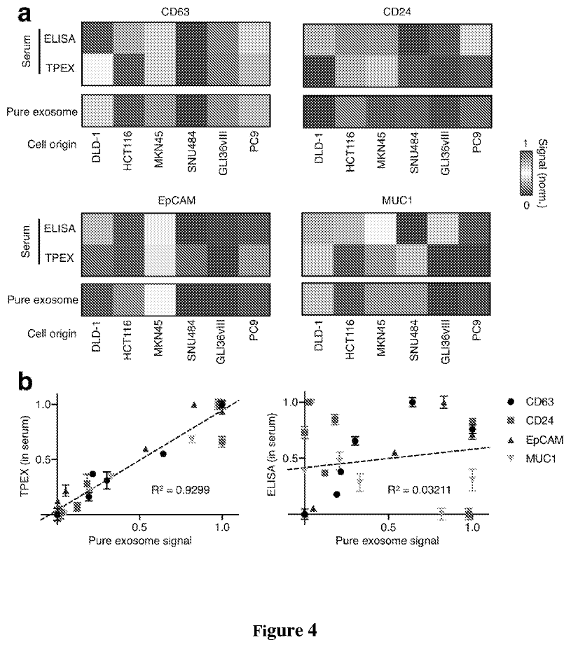

For all serum-spiked samples, comparative analysis was also performed with conventional sandwich ELISA assays (see Table 1 for the list of aptamers and antibodies). For each marker analyzed, when compared to the pure exosome signatures (obtained from the identical exosomes before spiking), the TPEX analysis showed a better concordance to reflect the expression trends across cell lines (FIG. 4a). Specifically, the TPEX analysis of the spiked samples showed a good correlation (R²=0.9299, FIG. 4b, left) to the pure exosome signatures, while the ELISA measurements performed on the same spiked samples showed a significantly poorer correlation (R²=0.03211, FIG. 4b, right). This performance difference was attributed to TPEX's multi-selectivity (i.e., exosomal biophysical properties and biomarker compositions) in measuring exosomal markers directly against complex background. The ELISA analysis, however, is only marker-sensitive and could be susceptible to free-floating forms of the target proteins (e.g., unbound proteins in human plasma.

TPEX Classification of Clinical Prognosis

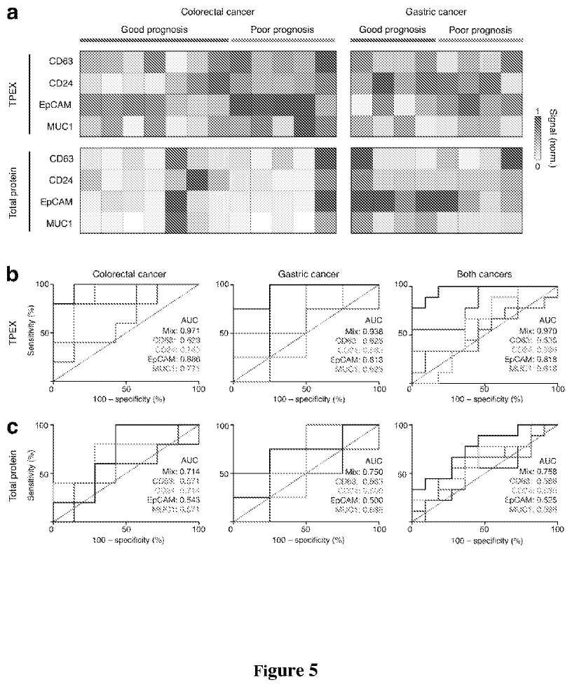

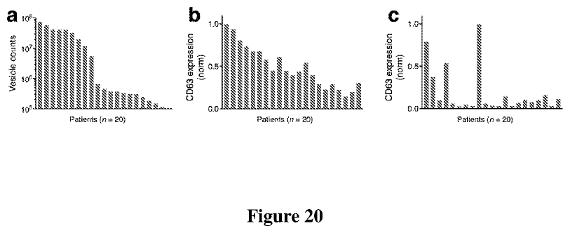

To evaluate the clinical utility of TPEX, a feasibility study using patient ascites samples was finally conducted. The aim was to address the following questions: (1) if TPEX could be directly applied to clinical specimens for multiplexed measurements, (2) the accuracy of TPEX in distinguishing exosomal targets, and (3) if the TPEX signatures could differentiate additional clinical characteristics (e.g., prognosis). Cancer ascites samples (n=20; 12 colorectal cancer and 8 gastric cancer) were obtained and the miniaturized microfluidic and detector platform (FIG. 1c-d) was used to perform multiplexed TPEX molecular analysis directly on these samples (1 μl for each native sample) (FIG. 5a, top). As a comparison, conventional, singleplex ELISA analysis was also performed to measure total target proteins in all clinical samples (FIG. 5a, bottom). Interestingly, the TPEX analysis (exosomal targets) showed different protein expression profiles to that measured by the ELISA analysis (total targets), consistent with published report. Across all clinical samples tested, the TPEX analysis of CD63 could reflect vesicle counts, as determined by gold-standard nanoparticle tracking analysis, while ELISA analysis of total CD63 proteins showed a poor concordance to the counts (FIG. 20).

Using individual patient survival data, as determined from the length of survival post ascites collection, the TPEX and ELISA measurements were used to develop regression scoring models for classification of disease prognosis. These models were validated using leave-one-out cross-validation and the performance of these models (mix) as well as individual markers through receiver operating characteristic (ROC) curve analysis (FIG. 5b-c) were compared. The TPEX model showed a higher accuracy in prognosis classification, across both cancer types (FIG. 5b, area under curve (AUC)=0.970), while the ELISA analysis of total target proteins showed a lower accuracy (FIG. 5c, AUC=0.758). This improved TPEX performance was attributed to the following reasons. Ascites contain target protein markers in different organizational states (e.g., exosome-bound and unbound). Recent studies have shown that these proteins are released through different mechanisms and play different roles in disease progression, highlighting the potential utility of exosomes as a more reflective indicator of disease aggressiveness and poor prognosis. Specifically, while free-floating membrane proteins are generally released during cell death, exosomes are secreted during active tumor growth and carry multiple cargoes to promote metastasis. TPEX's ability to distinguish and measure these reflective vesicle indicators could thus facilitate better disease stratification and prognostication.

### Discussion

Exosomes play an important role in mediating disease progression. Amongst other heterogeneous circulating factors found in bodily fluids, their orchestrated release by actively dividing cancer cells as well as functional activities in conditioning tumor microenvironment highlight the clinical potential of exosomes as a more reflective biomarker. Despite these recent discoveries, direct and specific analysis of exosomes in native clinical specimens remains challenging, due to limitations of existing analytical approaches. Specifically, exosomes are distinguished by unique biophysical and biomolecular properties; current detection of the exosome population, however, relies primarily on either biophysical or biochemical characterization, performed in an independent or sequential manner. Such analysis not only tend to miss vesicle subpopulations, but also fail to provide simultaneous, multiparametric analysis of vesicle biophysics and biomolecular composition.

To overcome these challenges, the TPEX platform was developed as a dedicated analytical platform for multi-selective molecular profiling of exosomes directly in clinical samples, through simultaneous and in situ evaluation of biophysical and biochemical compositions of the same vesicles. The technology is well-suited for rapid and multiparametric analysis of exosomes: (1) the assay design is multi-selective, for exosome biophysical properties (e.g., membrane envelope and characteristic dimensions) and co-localized biomolecular contents of the same vesicles; (2) the technology can be adapted to measure diverse exosomal biomarkers (e.g., proteins and miRNAs), but remain unresponsive to non-vesicle, free molecules; and (3) its implementation with the smartphone-based sensor not only enables multi-modal analysis (e.g., absorbance and fluorescence), but also streamlines the assay process to obviate any washing steps. The entire assay can be completed in as little as 15 minutes, while requiring 1 μl of native sample. Employing the developed technology, it was demonstrated that the TPEX platform could distinguish biomarker organizational states (i.e., exosome-associated vs. total biomarkers) and that the exosomal subpopulation of biomarkers could more accurately differentiate cancer patient prognosis.

The scientific applications of the developed technology are potentially broad. With its robust ability to differentiate biomarker organization in native clinical samples, the TPEX technology could be readily expanded to measure other molecules and modifications, and investigate their incorporation and/or association with diverse vesicles. Since the nanoshell growth is templated by vesicle biophysics, its plasmonic properties could be tuned to measure other extracellular vesicles of distinct sizes (e.g., oncosomes) and molecular subtypes (e.g., derived from different cell origins). Further technical improvements through incorporating other molecular probes and advanced recognition mechanisms could improve the analytical performance of the technology to measure even rare and complex molecular modifications. Such studies will not only facilitate comprehensive vesicle characterization, but also provide additional insights about compositional changes of secreted factors during disease progression.

The technology could also be developed and adapted for diverse clinical benefits. Specifically, the TPEX platform could be applied to discover new biomarker signatures and refine existing clinical biomarkers, through the incorporation of multiparametric analysis of biomarker organization, vesicle biophysics and molecular composition. Such developments will not only distinguish biomarker subpopulations, but could also shed light on the biophysical and/or biochemical properties of the associated biomarkers, thereby providing a new avenue to establishing accurate composite signatures. For clinical translation, the TPEX platform is fast, sensitive and wash-free. With its demonstrated robustness in native patient specimens, the system could be applied to various clinical samples (e.g., serum, urine) across a spectrum of diseases (e.g., cancers, neurodegenerative diseases). Further technical improvements, such as multiplexed microfluidic compartmentalization and array-type sensor integration, could enable highly parallel detection and facilitate large-scale clinical validation.

1. A method for detecting and/or characterising a nanovesicle in a sample, the method comprising the step of:
a) contacting a sample with nanoparticles or a precursor thereof, wherein the nanoparticles or precursor are capable of binding onto the surface of a nanovesicle and form, in situ, a nanoshell that surrounds said nanovesicle,
b) irradiating the sample and measuring the optical signals of the sample to detect and/or characterise the nanovesicle in the sample.

2. The method of claim 1, wherein the formation of the nanoshell induces a localized plasmonic resonance and/or increased optical absorbance in the infrared region.

3. The method of claim 1 or 2, wherein the spectral properties (absorbance) of the nanoshell are tuned to distinguish the nanovesicle dimension and the respective vesicle counts.

4. The method of any one of claims 1 to 3, wherein the nanoparticles are metallic nanoparticles.

5. The method of claim 4, wherein the metallic nanoparticles are gold nanoparticles.

6. The method of claim 5, wherein the metallic nanoparticles have a diameter range of between 7-11 nm.

7. The method of any one of claims 1 to 6, wherein the nanovesicle is an exosome.

8. The method of any one of claims 1 to 7, wherein the precursor is a metallic salt.

9. The method of any one of claim 8, wherein the metallic salt is gold salt.

10. A method for detecting one or more targets that are bound or associated with a nanovesicle in a sample, the method comprising the step of:
a) sequentially or simultaneously contacting a sample with nanoparticles or a precursor thereof and one or more fluorescent molecular probes, wherein the nanoparticles or precursor are capable of binding onto the surface of said nanovesicle and form, in situ, a nanoshell that surrounds said nanovesicle, and wherein the one or more fluorescent molecular probes are capable of specifically binding to one or more targets that are bound or associated with the nanovesicle and provide a unique emitting fluorescence wavelength for each said target,
b) irradiating the sample and measuring the emitted fluorescence in order to detect the one or more targets that are bound or associated with the nanovesicle, wherein the detection involves identifying an enhanced fluorescence quenching of the unique emitted fluorescence for each said target.

11. The method of claim 10, wherein the optical properties of the fluorescent molecular probes are matched to spectral-compatibility of the nanoshell to enhance the detection signal.

12. The method of claim 10 or claim 11, wherein the optical properties of the fluorescent molecular probes are matched to spectral-compatibility of the nanoshell to distinguish targets which reside within or associate with different-sized nanovesicles.

13. The method of any one of claims 10 to 12, wherein the one or more targets are selected from the group consisting of a protein, a nucleic acid, a lipid and a metabolite.

14. The method of any one of claims 10 to 13, wherein the nanovesicle is an exosome.

15. The method of any one of claims 10 to 14, wherein the nanoparticles are metallic nanoparticles.

16. The method of claim 15, wherein the metallic nanoparticles are gold nanoparticles.

17. The method of claim 16, wherein the metallic nanoparticles have a diameter range of between 7-11 nm.

18. The method of any one or claims 10 to 17, wherein the precursor is a metallic salt.

19. The method of claim 18, wherein the metallic salt solution is a gold salt.

20. The method of any one of claims 10 to 19, wherein the method involves contacting the sample with nanoparticles in excess required to form the nanoshell.

21. The method of any one of claims 10 to 20, wherein the fluorescent molecular probe is a nucleic acid, aptamer, peptide, antibody or small molecule.

22. The method of any one of claims 10 to 21, wherein the fluorescent molecular probe is modified with branched fluorescence to enhance detection signal.

23. The method of any one of claims 10 to 22, wherein the sample is a sample that has been obtained from a subject.

24. The method of claim 23, wherein the subject is a subject suffering from cancer.

25. The method of any one of claims 10 to 24, wherein the characterisation of the one or more targets comprises measuring the level of the one or more targets that are bound or associated with the nanovesicle.

26. A microfluidic chip for performing a method according to any one of the above claims.

27. A kit for performing a method according to any one of claims 1-25.

28. A method of determining the prognosis of a cancer in a subject by simultaneously detecting or characterising one or more targets that are bound or associated with nanovesicles in a sample from the subject and are indicative of the nature of the cancer, the method comprising:
a) sequentially or simultaneously contacting a sample with nanoparticles or a precursor thereof and one or more fluorescent molecular probes, wherein the nanoparticles or precursor are capable of binding onto the surface of said nanovesicle and form, in situ, a nanoshell that surrounds said vesicle, and wherein the one or more fluorescent molecular probes are capable of specifically binding to one or more targets that are bound or associated with the nanovesicle and provide a unique emitting fluorescence wavelength for each said target,
b) irradiating the sample and measuring the absorbance and/or emitted fluorescence in order to detect the one or more targets that are bound or associated with the nanovesicle, wherein the detection involves identifying an enhanced fluorescence quenching of the unique emitted fluorescence for each said target.

29. The method of claim 28, wherein the cancer is colorectal or gastric cancer.

30. The method of claim 28 or claim 29, wherein the sample is clinical cancer ascites.

31. The method of any one of claims 28 to 30, wherein the nanoparticles are metallic nanoparticles.

32. The method of claim 31, wherein the metallic nanoparticles are gold nanoparticles.

33. The method of any one of claims 28 to 32, wherein the precursor is a metallic salt.

34. The method of claim 33, wherein the metallic salt is a gold salt.

35. The method of any one of claims 28 to 34, wherein the one or more targets comprises a target selected from the group consisting of CD63, CD24, EpCAM and MUC1.


SEQUENCE LISTING (6 sequences)

SEQ ID NO: 1 - DNA, 32 bp, Artificial Sequence
  CD63 Aptamer
  misc_feature (32)..(32): modified wtih -NH2
caccccacctcgctcccgtgacactaatgcta

SEQ ID NO: 2 - DNA, 40 bp, Artificial Sequence
  CD24 Aptamer
  misc_feature (40)..(40): Modified with -NH2
tatgtgggtgggtgggcggttatgctgagtcagccttgct

SEQ ID NO: 3 - DNA, 48 bp, Artificial Sequence
  EpCAM Aptamer
  misc_feature (48)..(48): Modified with -NH2
cactacagaggttgcgtctgtcccacgttgtcatggggggttggcctg

SEQ ID NO: 4 - DNA, 25 bp, Artificial Sequence
  MUC1 Aptamer
  misc_feature (25)..(25): Modified with -NH2
gcagttgatcctttggataccctgg

SEQ ID NO: 5 - DNA, 22 bp, Artificial Sequence
  21-5p miRNA
  misc_feature (1)..(1): Modified with -NH2
tcaacatcagtctgataagcta

SEQ ID NO: 6 - DNA, 23 bp, Artificial Sequence
  221-3p miRNA
  misc_feature (1)..(1): Modified with -NH2
gaaacccagcagacaatgtagct